(19) United States
(12) Patent Application Publication
(10) Pub. No.: US 2007/0135691 A1
(43) Pub. Date: Jun. 14, 2007

(54) Medicament compliance monitoring system, method, and medicament container

(75) Inventors: Virginia Ann Zingelewicz, Scotia, NY (US); Steven Hector Azzaro, Schenectady, NY (US); Stephen Eric Zingelewicz, Scotia, NY (US); Austars Raymond Schnore JR., Scotia, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 11/301,230

(22) Filed: Dec. 12, 2005

(51) Int. Cl. A61B 5/00; A61M 31/00; A61B 5/02; A61B 5/103

(52) U.S. Cl. 600/301; 600/595; 600/485; 600/500; 604/66; 604/503

(57) ABSTRACT

A medicament compliance monitoring system and method of monitoring medicament compliance are disclosed. The medical compliance monitoring system includes a detector, a transmitter, and a monitoring center. The detector is configured to detect an activity indicative of medical compliance of a medicament. The transmitter is in communication with the detector. The monitoring center is in communication with the transmitter. The transmitter is adapted to communicate data from the detector to the monitoring center in real-time to the occurrence of the activity indicative of compliance. The method includes detecting an activity indicative of medical compliance of a medicament; and transmitting the detected activity to a monitoring center in real-time to the occurrence of the activity indicative of medicament compliance. Also disclosed is a medicament container.

### BACKGROUND

The invention includes embodiments that may relate to systems and method of monitoring medicament compliance. Particularly, the invention includes embodiments that may relate to systems and method of monitoring medicament compliance in real-time or near real-time.

To be effective as well as to make accurate scientific conclusions about a medicament's effectiveness, the medicament should be taken in a compliant manner, such as by a known or prescribed regimen. Taking a medicament (i.e. medicine, medication such as a drug) in a non-compliant manner may cause various problems. For example, many deaths may be related to non-compliance, many nursing home admissions may be related to non-compliance, and a main driver behind expenses of drug trials may also be related to non-compliance.

A variety of methods and systems may be known for monitoring medical compliance. However, some methods and systems may monitor medicament compliance in a delayed time, as opposed to in real-time or near real-time. Furthermore, the methods and systems may rely on the subject to accurately report his or her own compliance, which may have a delay of maybe even months and may be inaccurate. Some methods and systems may monitor medicament compliance by detecting an isolated individual instance or activity in a delayed time.

Thus, methods and systems of monitoring a patient's medication compliance are still needed.

### BRIEF DESCRIPTION

The embodiments of the invention will be set forth and apparent from the description that follows, as well as will be learned by practice of the embodiments of the invention. Additional aspects will be realized and attained by the methods and systems particularly pointed out in the written description and claims hereof, as well as from the appended drawings.

Accordingly, an aspect of the invention includes a medicament compliance monitoring system. The medicament compliance monitoring system includes a detector, a transmitter, and a monitoring center. The detector is configured to detect an activity indicative of medical compliance of a medicament. The transmitter is in communication with the detector. The monitoring center is in communication with the transmitter. The transmitter is adapted to communicate data from the detector to the monitoring center in real-time to the occurrence of the activity indicative of compliance.

Another aspect of the invention includes a method of monitoring medicament compliance. The method includes detecting an activity indicative of medical compliance of a medicament by a subject; and transmitting the detected activity to a monitoring center in real-time to the occurrence of the activity indicative of medicament compliance.

Another aspect of the invention includes a method of monitoring medicament compliance of a subject. The method includes providing an activity detector; collecting data from the activity detector; and providing a transmitter in communication with the detector. The activity is indicative of medical compliance of a medicament. The transmitter communicates the data from the activity detector to a monitoring center in real-time to the occurrence of the activity indicative of medicament compliance.

Another aspect of the invention includes a medicament container. The medicament container includes a compartment configured to hold a medicament. The compartment includes a sensor configured to detect removal of the medicament.

Another aspect of the invention includes a medicament compliance monitoring system. The medicament compliance monitoring system includes a plurality of detectors and monitoring center. The plurality of detectors are configured to detect a plurality of activities indicative of medical compliance of a medicament. The monitoring center receives data from the plurality of detectors in real-time to the occurrence of the plurality of activities indicative of compliance.

The accompanying figures, which are incorporated in and constitute part of this specification, are included to illustrate and provide a further understanding of the method and system of the invention. Together with the description, the drawings explain the principles of the invention.

### BRIEF DESCRIPTION OF THE DRAWINGS

FIG. 1 is a schematic representation of a medicament compliance monitoring system in accordance with an embodiment of the invention;

FIG. 2 is another schematic representation of a medicament compliance monitoring system in accordance with an embodiment of the invention;

FIG. 3 is a flow chart of a method of monitoring a medicament compliance in accordance with an embodiment of the invention; and

FIG. 4 is another flow chart of a method of monitoring medicament compliance in accordance with an embodiment of the invention.

### DETAILED DESCRIPTION

Exemplary embodiments of the invention are illustrated in the accompanying figures and examples. Referring to the drawings in general, the illustrations describe a particular embodiment of the invention and do not limit the invention thereto.

Whenever a particular embodiment of the invention is said to comprise or consist of at least one element of a group and combinations thereof, it is understood that the embodiment may comprise or consist of any of the elements of the group, either individually or in combination with any of the other elements of that group. Furthermore, when any variable occurs more than one time in any constituent or formula, its definition on each occurrence is independent of its definition at every other occurrence. Also, combinations of substituents and/or variables are permissible only if such combinations result in stable systems and compositions.

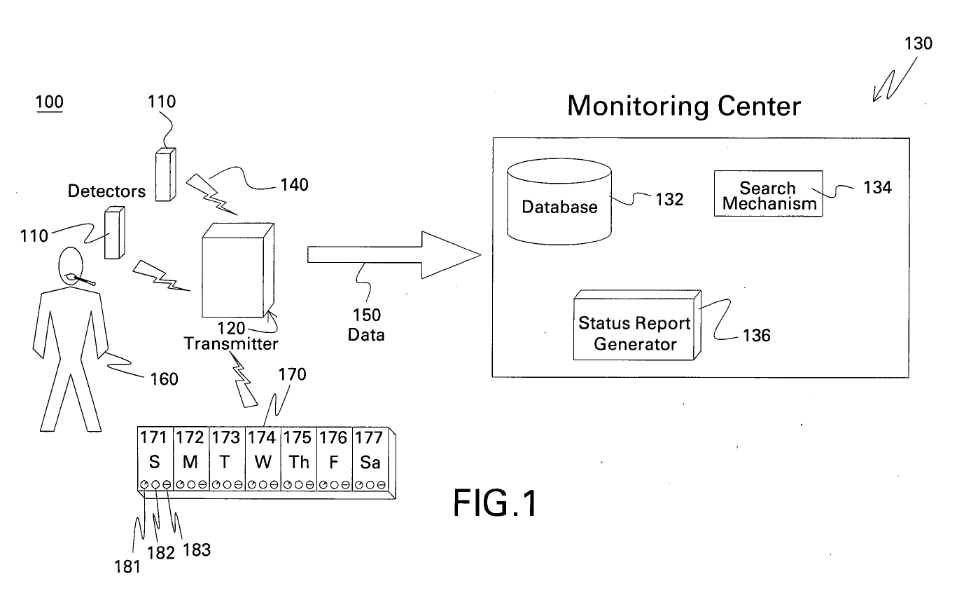

Medicament compliance monitoring systems and methods of monitoring medicament compliance are disclosed. One embodiment of a medicament compliance monitoring system 100 is shown in FIG. 1. The medicament compliance monitoring system includes one or more detectors 10, one or more transmitters 120, and a monitoring center 130. Unless noted otherwise, the medicament includes any substance such as a drug to cure, treat, or prevent a disease or condition. Medicament may also include and be referred as medicine or medication.

The detector is configured to detect an activity that is indicative of medical compliance of a medicament. The transmitter 120 is in communication 140 with the detector. The transmitter 120 is adapted to communicate data 150 from the detector to the monitoring center 130 in real-time to the occurrence of the activity indicative of compliance. Real-time includes a range from almost instantaneously up to about 60 minutes from occurrence of the activity. In one embodiment, real-time includes a range of 1-30 seconds, 30-60 seconds, 1-5 minutes, 5-10 minutes, 10-15 minutes, 15-20 minutes, 20-25 minutes, 25-30 minutes, and 30-35 minutes from the occurrence of the activity.

In one embodiment, the detector is on a subject 160 or within a vicinity of a subject 160, as shown in FIG. 1. Examples of detectors on a person include a calorimeter, pulse rate, motion detector, heart rate monitor, and motion actigraphy. The range of “within a vicinity” may vary based on the activity and the detector. In one embodiment, within a vicinity includes ranges such as 1-100 feet, 1-80 feet, 1-70 feet, 1-50 feet, 1-30 feet, 1-20 feet, 1-5 feet, 1-10 feet, 2-3 feet, 1-2 feet, and 1-2 inches. The subject may be a patient, for an existing condition or preventive condition. Examples of such subjects include mammals, such as people. Other suitable mammals include such as, but not limited to, rats, pigs, etc. Examples of such subjects also include other animals besides mammals. In one embodiment, the patient may wear the detector and the detector may be configured to detect the activity when the patient is mobile, such as a pulse rate monitor, motion detector, heart rate monitor, and motion actigraphy.

The detector may include, but are not limited to, a motion detector, blood pressure detector, heart rate detector, medicament access detectors, chemical substance detector, sleep detector, weight loss or gain detector, pulse rate detector, and urinalysis detector, either individually or a combination of two or more thereof. The detector may be selected based on the particular activity to be detected, taking into account the patient condition and symptoms associated with a medicament. In one embodiment, the monitoring system includes a plurality of differing detectors. The plurality of differing detectors may detect the same or similar activities or differing activities. For example, the plurality of differing detectors may detect an activity such as weight change or the plurality of differing detectors may detect multiple differing activities, such as a weight change or pulse rate.

Examples of activities indicative of compliance with a medicament include, but are not limited to, a physiological aspect, a side effect, a disease abatement, and a physical indicator, either individually or a combination of two or more thereof. Unless noted otherwise, “indicates or indicative of compliance” includes indications of compliance or lack thereof. The categories of the descriptions of activities may overlap and is for illustration and not limitation. Activity includes an activity or a condition or state of being.

Non-limiting examples of physiological aspects include an excreted form of a medicament such as a drug, an excreted drug-specific metabolite, an exhaled form of a drug, and exhaled particulates, either individually or a combination of two or more thereof. Real-time is measured from the occurrence of the activity that is indicative of the medicament compliance, not from the administration of the medicament. For example, the medicament may take hours to have a physiological aspect such as being excreted, but the transmission is in real-time from the occurrence of the activity being detected, such as a physiological activity of excretion.

Examples of side effects include, but are not limited to, coughing, swelling of lower legs, swelling of feet, low pulse, tiredness, difficulty sleeping, weight gain, frequent urination, tiredness, dehydration, loss of appetite, sore throat, sleeping difficulties, increased heart rate, dry mouth, decreased appetite, dry throat, fatigue, and upset stomach, either individually or a combination of two or more thereof.

Examples of disease abatement include stabilization of a condition, lack of shortness of breath while resting, changes in sleep, changes in eating, reduced heart rate, elimination of excess fluids, maintenance of constant weight, improved lung function, improved symptoms, reduced acute symptom, reduced hospitalization need, decreased use of rescue inhaler, and reduction of acute asthma attack, either individually or a combination of two or more thereof.

Examples of physical activities that are indicative of medicament intake include physical moving of a medication, opening of a medicament container, moving of a medicine bottle, running of water, cup movement, and signs of drug disposal such as a toilet flush or an increase in weight of a garbage pail, either individually or in a combination of two or more. Physical moving of a medication may be detected by various types of motion detectors. Opening of a medicament container may be detected by a medicament container configured with sensors. Moving of a medicine bottle may be detected by various motion detectors. Running of water may be detected by a motion detector attached to the faucets. Cup movement may be detected by motion detectors. Signs of drug disposal may be detected by a toilet flush detector or a motion detector for a garbage pail opening.

In some embodiments, detection of a single activity, such as the excreted form of a drug or drug-specific metabolite, indicates compliance of a medicament. In fact, the excreted form of a drug or drug-specific metabolite may indicate compliance of a particular medicament and only that medicament such that there is a one to one correlation between the activity and the medicament. In other embodiments, a single activity, such as increased or decreased heart rate, may correspond to or indicate compliance of various medicaments, such that there is not a one to one correlation between a given activity and a medicament. Although a single activity could, but may not necessarily, indicate compliance of a particular medicament, various kinds of detectors may detect multiple combinations of activities, the combination of which are indicative of medical compliance of a medicament. Detection of a combination of multiple activities may also be useful if one activity cancels or diminishes the detectability of another activity. Thus, the system includes multiple detectors configured to detect multiple activities indicative of medical compliance of a medicament. Furthermore, the multiple activities indicative of medicament compliance may correlate to a medicament or a plurality of medicaments that differ from each. For example, as shown in Table 1, the infra, detectors may detect multiple activities that correspond to an individual medicament, such as an angiotensin converting enzyme (ACE) inhibitor. Several kinds of detectors may detect multiple activities that correspond to compliance of taking an ACE inhibitors such as coughing which may be detected by a noise detector, swelling of lower legs and feet which may be detected by a pressure monitor, stabilization of condition, shortness of breath while resting, changes in sleep or eating which may be detected by a motion detector, an excreted drug and excreted drug-specific metabolites may be monitored by urine sample, an exhaled drug or particulates, either individually or in combinations of two or more. Thus, although a single activity may not necessarily indicate compliance, the various kinds of detectors may detect multiple combinations of activities that are indicative of medical compliance of a medicament, such as an ACE inhibitor. A weight monitor detector may detect the weight gain or loss, which may also detect changes in eating, weight gain or loss, loss of appetite, and maintenance of constant weight. A detector like a heart rate monitor may detect the pulse or heart rate or a change in heart and pulse rate. A detector-like a blood pressure monitor may detect blood pressure and change in blood pressure over a time interval.

In another example, several kinds of detectors may detect multiple activities that correspond to compliance of taking a diuretic, such as, but are not limited to, rapid weight gain which may detected by a weight scale, frequent urination which may be detected by detectors on a toilet flush handle and or toilet bowl, extreme tiredness which may be detected by motion detectors, dehydration which may be detected by patches, loss of appetite, stabilization of condition, shortness of breath while resting, changes in sleep or eating which may be detected by motion detector, an excreted drug or excreted drug-specific metabolites which may be monitored by urine sample, and an exhaled drug or particulates, either individually or in combinations of two or more. Thus, although a single activity may not necessarily indicate compliance, the various kinds of detectors may detect multiple combinations of activities, the combination of which are indicative of medical compliance of a medicament, such as a diuretic.

Furthermore, the detectors may detect multiple activities that correspond to differing medicaments for a single medical condition or multiple medical conditions that differ from each other. For example, the detectors may detect activities indicative of a single medical condition like CHF (congestive heart failure) or multiple medical conditions that differ from each other, such as CHF and asthma.

The following table A lists some non-limiting examples of activities and detectors that may detect the activities.

TABLE A
Activity or Condition being detected--Detector

| Activity or condition | Detector |
|---|---|
| coughing | noise detector |
| swelling of lower legs and feet | pressure detector |
| changes in sleep | motion detector or a pressure pad in pad |
| changes in eating | motion detector, detector on refrigerator, |
| | stove, and or cabinets |
| slow or high pulse | pulse rate monitor |
| tiredness | motion or activity monitor |
| weight gain or loss | weight scale and or detector on refrigerator, |
| | stove, and or cabinets |
| difficulty sleeping | activity monitor for evening or night |
| frequent urination | detector on toilet bowl or flush or toilet paper |
| dehydration | patch detector for sweat |
| tiredness | motion detectors, pulse rate, heart rate, |
| loss of appetite | motion detector in food preparation or |
| | kitchen area, detector on refrigerator, stove, |
| | and or cabinets, |
| the excreted drug or drug-specific | urine sample detector |
| metabolites excreted | |
| for inhaled drugs: exhaled drug or exhaled | breath detector |
| particulates | |

The activity that is indicative of medicament compliance may indicate whether the patient is compliant with a dosage intake, a dosage amount, and or dosage frequency, either individually or a combination of two or more thereof. In one embodiment, the activity indicative of medicament compliance indicates whether the patient is compliant with a dosage intake. In another embodiment, the activity indicative of medicament compliance indicates whether the patient is compliant with a dosage amount. In yet another embodiment, the activity indicative of medicament compliance indicates whether the patient is compliant with a dosage frequency. Also, one or more activities may indicate whether the patient is compliant with a dosage intake, a dosage amount, and or dosage frequency of multiple medicaments. Furthermore, the one or more activities may indicate whether the patient is compliant as to the dosage intake, the dosage amount, and or the dosage frequency, either individually or a combination of two or more thereof, for the multiple medicaments.

The transmitter 120 may be adapted to communicate data 150 from the detector 110 to the monitoring center 130 in real-time to the occurrence of the activity indicative of compliance. In some embodiments, the detector and the transmitter may be the same entity, wherein the detector is also a transmitter. Real-time includes in the range of almost instantaneously up to about 60 minutes from occurrence of the activity. In one embodiment, real-time includes a time range from about 1 to about 30 seconds, a time range from about 30 to about 60 seconds, a time range from about 1 minute to about 5 minutes, a time range from about 5 minutes to about 10 minutes from occurrence of the activity. Examples of transmitters include communication media such as, but not limited to, wired telephone, wireless telephone, two-way walkie-talkie, pager, cable, and the Internet, either individually or a combination of two or more thereof. Depending upon the transmitter chosen, the data may be sent in real-time at a predetermined interval, such as discrete or continuous interval, regular or irregular intervals. For example, the data may be sent in real-time via wireless telephone, two-way walkie-talkie, pager, cable, the Internet or any other wired or wireless communication platform. For a telephone communication platform, the data signals may be buffered and transmitted at differing intervals.

In one embodiment, the monitoring center 130 includes a database 132 for receiving and compiling the data on activities indicative of medicament compliance. The monitoring center may be adapted to communicate with a caregiver through one or more communication media such as wired telephone, wireless telephone, pager, two-way walkie-talkie, facsimile, cable, e-mail, and the Internet.

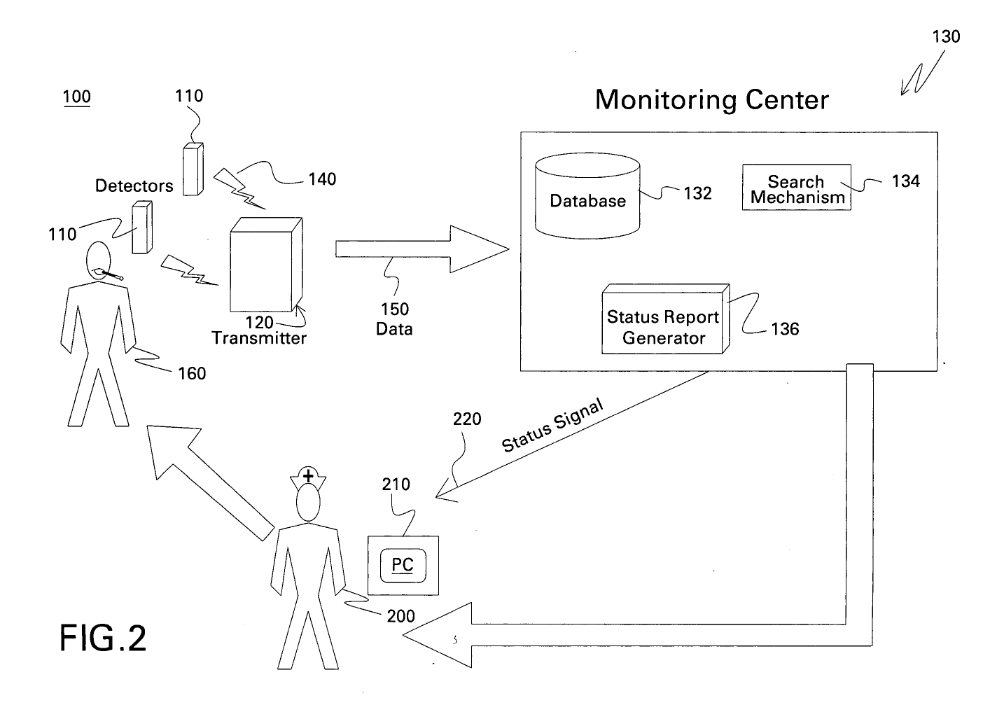

The monitoring center 130 may be remote from the patient as shown in FIG. 1 and FIG. 2. The monitoring center may include a database 132, a search mechanism 134, and a status report generator 136. The database serves as a collection vessel for the data generated by the detector. The search mechanism may be adapted to search for patterns in the activities indicative of medicament compliance of one or more patients and make a conclusion about compliance. The monitoring center may be programmed or configured to conduct various models on the cumulative collected data of the detected activities and make a conclusion about compliance. Examples of models include artificial intelligence, statistical modeling, and hybrid fusion techniques, either individually or in a combination of two or more. Non-limiting examples of statistical modeling include Bayesian Belief Networks and case-based reasoning. When a single activity may not necessarily indicate compliance, multiple combinations of activities detected by various kinds of detectors may be indicative of medical compliance of a medicament. The models may take into account historical tracking and trends. The models may indicate a qualitative yes or no answer of compliance or a quantitative answer of degrees of compliance. For example, the models may indicate degrees of compliance in a time unit of minutes from a given dose time; the models may indicate degrees of compliance in a weight unit of milligram from a dose amount. Thus, the monitoring system may make conclusions about compliance with a dosage amount and or a dosage frequency in a qualitative manner, such as yes or no, or in a quantitative manner, such as degrees of compliance with dosage amount and or dosage frequency. For example, the monitoring system may conclude in a yes or no manner that the dosage amount and or dosage frequency was not taken correctly for CHF. The monitoring system may also indicate that the dosage amount and or dosage frequency was not taken correctly by how much quantity or time.

In one embodiment, the system may include a communications relay panel positioned within vicinity of the patient and in communication with the detector and the transmitter.

As described in FIG. 2, upon a request for a status report, such as from a caregiver, the data is forwarded from the database 132 to the status report generator 136. Examples of caregivers include, but are not limited to, a nurse, doctor, or a family member. The status report generator 136 may communicate a status signal 220 to a personal computer 210 of the caregiver 200. The status signal may be in real-time or substantially simultaneous with the status report being generated. Substantially simultaneous includes anywhere in the range of almost instantaneously to up to fifteen minutes. For example, for a two-way page communication platform the amount of time required for the communication can be between two and three minutes. The status report generator may be programmed to update the report for a patient at a predetermined interval, such as, for example, every ten minutes. The format and substance of the report are dependent upon the request of the caregiver and may be adjusted accordingly. It should be appreciated that the signal can instead be communicated via a personal digital assistant (PDA), a pager, a facsimile machine, cable, or a telephone or voice-mail account instead of via the personal computer 210.

The medicament monitoring system may be adjusted or programmed to determine which activity is indicative of medicament compliance. Furthermore, the parameters of what activity are indicative of medicament compliance, such as in a quantitative or qualitative manner may be adjusted. What constitutes compliance or non-compliance, or degrees of compliance may be chosen or programmed from a set of predefined activities. Further, the parameters of an activity may be configured to match the normal or baseline activity of a specific individual patient. For example, what constitutes “heart rate” may be known, but when “a heart rate” would be indicative of medical compliance with a medicament may be programmed. The data is stored and processed at the monitoring center. Alternatively, unprogrammed changes may be detected as abnormalities for a particular patient. Such unprogrammed changes or abnormalities may be detected using adaptive models based on the previously mentioned algorithms. If the data indicates the occurrence of an activity that is indicative of medical compliance, a signal is sent to the caregiver via any suitable communication medium, such as, for example, wired or wireless telephone, PDA, pager, facsimile, cable, two-way walkie-talkie, e-mail, or other Internet-supported communication media. The caregiver may communicate with the patient. The communication may be through a communication pathway, such as a wired or wireless telephone line, the Internet (i.e., e-mail or other Internet-based communication tool), cable, PDA, pager, or personal, such as a visit by the caregiver or another suitable person.

Also described is an embodiment of a medicament container 170 for holding the medicament, as shown in FIG. 1. The medicament container is configured to detect removal of the medicament from the medicament container. The medicament container 170 for may have various compartments, 171-177, for each day, like Sunday to Saturday to hold one or more medicaments. The medicine container may have different compartments for dosing medication. For example, compartments may be BID (twice a day), TID (three times a day), and QID (four times a day). The compartment may include a cover and include one or more sensors configured to detect removal of the medicament. The medicament container may vary in size, shape, and material. In one embodiment, the medicament container is made from plastic. Other materials include ceramic, wood, or metal, either individually or in combinations thereof. Each compartment of the medicament container may vary in size, shape, and material to accommodate medicaments of varying shape and form, such as solid, pill, tablet, powder, or liquid. In one embodiment, the compartment is configured to accommodate a solid form such as pill, tablet, gel, or powder.

The medicament compliance monitoring system may also include the medicament container. In one embodiment, the Sunday to Saturday medicament container may be fitted with a circuit capable of or configured to detect when any of the compartments has been opened. The medicament container may include a micro that indicates which compartment has been opened. As shown in FIG. 1, the medicament container may be in communication with the transmitter 120, which can transmit to the monitoring center, where the data will be analyzed and stored. In one embodiment, when the patient is home or within a vicinity of a transmitter, the container micro may transmit in real-time when a compartment has been opened. Real-time is measured from the occurrence of the activity that is indicative of the medicament compliance, not from the administration of the medicament. In this case, the activity would be the opening of the compartment when the patient is home or within a vicinity of a transmitter. Real-time includes a range from almost instantaneously up to about 60 minutes from occurrence of the activity. In one embodiment, real-time includes a range of 1-30 seconds, 30-60 seconds, 1-5 minutes, 5-10 minutes, 10-15 minutes, 15-20 minutes, 20-25 minutes, 25-30 minutes, and 30-35 minutes from the occurrence of the activity.

The circuitry to detect the opening of a compartment may include any number of implementations. The circuit is capable or operable is to indicate a change in the state of the medicament container, record the time interval of such state change, and to transmit an immediate or logged record of the state change. In addition, continuous information maybe recorded for transmission. This may include location GPS (global positioning system), temperature, attitude (rotation position), acceleration, humidity, light intensity, and medication weight.

In one embodiment, recorded information is stored in a microprocessor. Information may also be captured via ASIC (application specific integrated circuit) or other devices. This recorded information is transmitted to a base station and then dispersed via any number of methods to a monitoring center. Furthermore, at designated intervals, such as every 4 hours, a log (i.e. storage of information) may be transmitted that summarizes all the transmissions in the previous interval. The log may allow transmissions that did not go through, such as for example, if the patient was away from home or an error. The log may also allow any transmissions that were not received by the server to be re-transmitted. If a patient takes a medicament from the medicament container when away from home and not near a transmitter, compliance may be measured from the time the patient returns home and or is within a vicinity of a transmitter.

A receiver in the patient's home may be able to distinguish between multiple medicament containers, thus allowing a caregiver to distinguish between patients by a separate, monitored medicament container.

In one embodiment, the medicament container is configured to detect removal of the medicament by detecting changes such as, but not limited to, a change in a weight of the medicament container, shape, size, and color, either individually or a combination of two or more.

For example, in one embodiment, the medicament container may be configured to detect removal of the medicament by detecting a change in a weight of the medicament container. As shown in FIG. 1, a miniature weight scale 181 in the bottom of a compartment will allow the weight after a medicament is removed from the compartment to be transmitted in correlation with the time the compartment was opened. The miniature scale may also detect the difference in weight for a plurality of medicaments that are removed.

In another embodiment, the medicament container may detect the removal of the medicament by detecting the colors of the medicaments remaining in a compartment after a medicament(s) is removed in correlation with the time the compartment was opened. As shown in FIG. 1, the medicament container may include one or more light sensors 182 in each compartment that detects the color of the remaining medicaments in the compartment after the compartment is opened.

In another embodiment, the medicament container may detect the removal of the medicament by detecting the presence of the medicaments remaining in a compartment after a medicament(s) is removed in correlation with the time the compartment was opened. As shown in FIG. 1, the medicament container may include one or more light sensors 183 in each compartment that detects the presence of the remaining medicaments in the compartment after the compartment is opened. The presence may be sensed by shape or size, etc.

The light sensor(s) 182,183 that senses the color or presence of the remaining medicament may be used in addition to or in replacement of the weight scale sensor 181.

The medicament container may detect removal of multiple medicaments that differ from each other. In one embodiment, the medicament container is configured to detect removal of the plurality of medicaments that differ from each other by detecting a combination or permutation of changes, such as, but not limited to, a change in a weight of the medicament container, shape, size, and color that correlate with the combination of medicaments.

The monitoring system as well as the detector and transmitter are not limited by the types of medicaments nor the form and frequency in which the medicaments are administered. The detector can be configured to detect an activity that is indicative of medical compliance of various types of medicaments as well as medicaments administered in various forms. The medicament may be a chemical and or a physical agent or a chemical and or a physical treatment. For example, the medicament may be the administration of an agent (e.g., an anti-inflammatory agent and/or an antiproliferative agent) and/or the application of a treatment (e.g., radiation therapy or surgery) intended to cure or ameliorate the symptoms of an inflammatory condition. Medicament includes therapeutic treatment as well as prophylactic or preventative measures. Furthermore, the detector may detect medical compliance of a medicament for a subject prone to having the disorder or diagnosed with the disorder or those in which the disorder is to be prevented. The detector may detect medical compliance of a medicament that is administered in various frequencies, such as consecutive treatment (administration refers to treatment on at least a daily basis without interruption in treatment by one or more days). Intermittent treatment or administration refers to treatment that is not consecutive, but rather cyclic in nature. The treatment regime herein can be either consecutive or intermittent.

The system is not restricted by the types of medicaments nor the form and frequency in which the medicament is administered. The medicament may be delivered orally, topically, parenterally, by inhalation spray, rectally, subcutaneous injections, intravenous, intramuscular, intrasternal injection or infusion techniques in dosage unit formulations.

The medicament can be administered in the form of a depot injection or implant preparation. The medicament may be formulated in such a manner to permit a sustained release of an active ingredient, which may be detectable. The medicament can also be administered in the form of liposome delivery systems, such as small unilamellar vesicles, large unilamellar vesicles and multilamellar vesicles. The medicament can also be administered in intranasal form via topical use of suitable intranasal vehicles, or via transdermal routes, using those forms of transdermal skin patches well known to those of ordinary skill in that art. To be administered in the form of a transdermal delivery system, the dosage administration may be continuous rather than intermittent throughout the dosing regimen.

The medicaments can also be co-administered by coupling with or paired with other agents suitable for a respective disease state or condition. The coupling of the medicament with an agent may enhance detectability of the medicament or the agent itself may be therapeutic or beneficial. For example, the medicament may also be delivered by coupling with or paired with monoclonal antibodies as individual carriers. The medicament may also be coupled with soluble polymers as targetable drug carriers. The dosage regimen utilizing the medicaments may be selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or other caregiver can readily determine and prescribe the effective amount of the medicament required to prevent, counter, or arrest the progress of a condition.

In other instances, for oral administration in the form of a tablet or capsule, the medicaments can be combined with an oral, non-toxic, pharmaceutically acceptable, inert carrier to enhance detectability of the medicament. Examples of an oral, non-toxic, pharmaceutically acceptable, inert carrier include lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water and the like. Moreover, when desired or necessary, suitable binders, lubricants, distintegrating agents and coloring agents can also be incorporated into the mixture. Suitable binders include starch, gelatin, natural sugars such as glucose or beta-lactose, corn-sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch methylcellulose, agar, bentonite, xanthan gum, and the like.

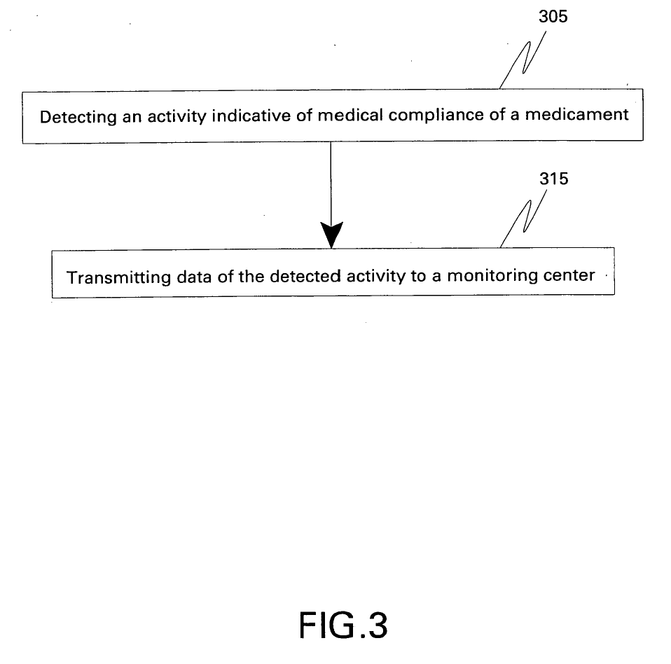

With reference to FIG. 3, a method of monitoring medicament compliance of a subject is described. FIG. 3 is a flow chart. Step 305 includes detecting one or more activities indicative of medical compliance of a medicament. Step 315 includes transmitting the detected activity to a monitoring center in real-time to the occurrence of the activity indicative of medicament compliance.

The detected activity may be correlated with medical compliance of one or more medicaments, as described herein above and in the examples below. Furthermore, the detected activity may be correlated with medical compliance of the intake of a medicament. The detected activity may be correlated with medical compliance of a dosage amount of a medicament. The detected activity may be correlated with medical compliance of a dosage frequency of a medicament. The method also includes detecting multiple activities that are indicative of medical compliance of a medicament. The method is not limited by when the multiple activities are detected. The multiple activities may be detected simultaneously or sequentially, and with various designated intervals of time. Furthermore, the activities may also be detected by one or more detectors.

The multiple detected activities may be correlated with medical compliance of a medicament, as described herein above and in the examples below. Furthermore, the detected activities may be correlated with medical compliance of a plurality of medicaments that are different from each other. In one embodiment, the detected activities may be correlated with medical compliance of intaking of the plurality of differing medicaments. In another embodiment, the detected activities may be correlated with medical compliance of the dosage amounts of the plurality of differing medicaments. In yet another embodiment, the detected activities may be correlated with medical compliance of the dosage frequencies of the plurality of differing medicaments.

In another embodiment, the plurality of detected activities may be correlated with medical compliance of dosage frequencies and dosage amounts of the plurality of medicaments that are different from each other, as described herein above and in the examples below.

The method includes monitoring medical compliance of a medicament such as, but not limited to, ACE Inhibitors, Beta-Blockers, Diuretics, Corticosteroids, short-acting Bronchodilators, long-acting Bronchodilators, and Statins, either individually or in a combination of two or more.

The method also includes monitoring medical compliance of a medicament capable of treating a condition such as, but not limited to, asthma, cholesterol, and congestive heart failure (CHF). The medical conditions may be similar or different from each other.

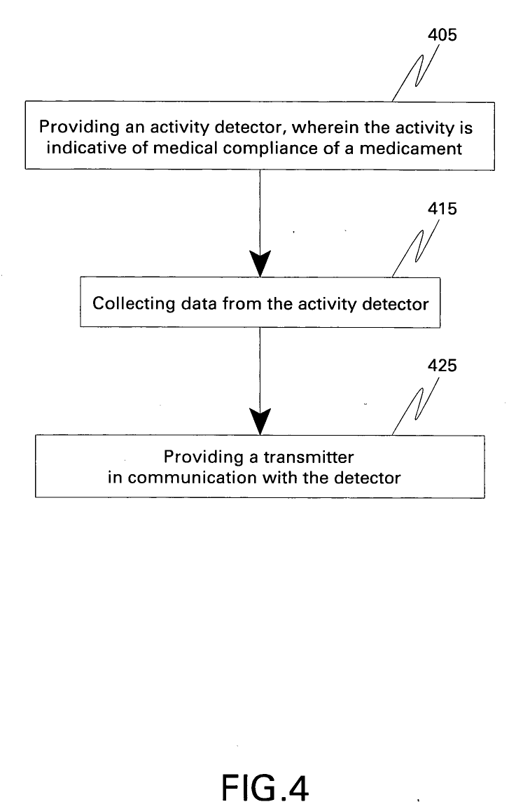

With reference to FIG. 4, another method of monitoring medicament compliance of a subject is described. FIG. 4 is a flow chart. Step 405 includes providing an activity detector. Step 415 includes collecting data from the activity detector and Step 425 includes providing a transmitter in communication with the detector. The activity is indicative of medical compliance of a medicament. The transmitter communicates the data from the activity detector to a monitoring center in real-time to the occurrence of the activity indicative of medicament compliance.

In one embodiment, the medicament is provided in a medicament container as described hereinabove. The medicament container is operable to detect removal of a medicament by detecting one or more change, such as a change in a weight of the medicament container, shape size and color. In one embodiment, the medicament container is configured to detect removal of a plurality of medicaments that differ from each other.

In a particular embodiment, the medicament container is operable to detect removal of the plurality of medicaments that differ from each other by detecting a combination of changes, such as a change in a weight of the medicament container, shape size and color.

Furthermore, a base-line pre-medicament measurement or detection, from a subject, of the activity being detected before administering the medicament may also be obtained. For example, if heart rate is the activity that is indicative of medical compliance, a base-line measurement or detection of the heart rate from a subject may be taken before administration of the medicament. The heart rate may then be detected by the detector after the medicament is administered. The base-line pre-medicament measurement of the detected activity, such as the heart rate, may then be compared with the measurement of the detected activity during or after the medicament is administered. The activity (such as the heart rate) may be repeatedly measured or detected at different time intervals as desired to evaluate medical compliance. One or more baseline pre-medicament measurement of a detected activity may be compared with measurement of the detected activity during or after the medicaments are administered or the additional during or after medication measurements may be compared with each other.

The method may also include taking measurement of the detected activity after being off a medicament, such as a post-treatment indication, and comparing the measurement after being off the medicament with other baseline measurements or measurements taken while on the medicament.

The method may further include comparing the post-treatment measurement or detection to the pre-treatment baseline measurement or detection to assess the effectiveness of the prescribed regimen.

The following 9 examples illustrate some features of the invention and are not intended to limit the invention thereto. Particularly, examples 1-3 (Table 1-3) and 6-8 (Table 6-8) demonstrate monitoring medical compliance by detecting multiple activities, the combination of which, indicate compliance of a medicament. Examples 4 (Table 4) and 8 (Table 8) demonstrate monitoring medical compliance by detecting multiple activities, the combination of which, indicate compliance with multiple medicaments for a given medical condition such as CHF or asthma, respectively. Example 9 demonstrates monitoring medical compliance by detecting multiple activities, the combination of which, indicate compliance with multiple medicaments for multiple medical conditions such as CHF and asthma, simultaneously.

Although the examples of medicament compliance describe the patient being human, a patient may also be an animal. For example, in one embodiment, the medicament compliance monitoring systems and methods may monitor medicament compliance of a pet being given a medicament, such as vaccinations and antibiotics, by a pet owner. The caregiver may be a veterinarian or any other caretaker of animals. The medicament compliance monitoring systems and methods may monitor medicament compliance of animals given a medicament on a large scale setting as well, such a pet shop or animal shelter, veterinarian office.

### EXAMPLE 1 ACE INHIBITORS

Example 1 shows monitoring medicament compliance by detecting multiple combinations of activities associated with compliance of an ACE Inhibitor medicament. As shown in Table 1, several kinds of detectors may detect multiple activities that correspond to compliance of taking ACE inhibitors, such as, but are not limited to, coughing which may be detected by a noise detector, swelling of lower legs and feet which may be detected by a pressure monitor, stabilization of condition, shortness of breath while resting, changes in sleep or eating which may be detected by motion detectors, and an excreted drug or excreted drug-specific metabolites which may be detected in an urine sample, either individually or in combinations of two or more. Thus, various kinds of detectors may detect the multiple activities indicative of taking an ACE inhibitor in a compliant manner.

### EXAMPLE 2 BETA-BLOCKER

Example 2 shows monitoring medicament compliance by detecting multiple combinations of activities associated with taking a Beta-blocker medicament. As shown in Table 2, several kinds of detectors may detect multiple activities that correspond to compliance of taking a Beta-blocker, such as, but are not limited to, a slow pulse which may be detected by a pulse rate detector or monitor, tiredness and difficulty sleeping which may be detected by motion detectors and or noise detectors, sudden weight gain which may be detected by weight scales, dehydration which may be detected by a patch, reduced heart rate which may be detected by a heart rate monitor, shortness of breath while resting which may de detected by a breath detector, changes in sleep or eating, and an excreted drug or excreted drug-specific metabolites which may be detected in an urine sample, either individually or in combinations of two or more. Thus, various kinds of detectors may detect the multiple activities, the combination of which is indicative of taking a Beta-blocker in a compliant manner.

### EXAMPLE 3 DIURETIC

Example 3 shows monitoring medicament compliance by detecting multiple combinations of activities associated with taking a diuretic. As shown in Table 3, several kinds of detectors may detect multiple activities that correspond to compliance of taking a diuretic, such, but are not limited to, rapid weight gain which may be detected by weight scales, frequent urination which may be detected by detectors on a toilet flush and or bowl, extreme tiredness which may be detected by motion detectors, dehydration which may be detected by a patch, loss of appetite, stabilization of condition, shortness of breath while resting, changes in sleep or eating which may be detected by motion detectors, and an excreted drug or excreted drug-specific metabolites which may be detected in an urine sample, either individually or in combinations of two or more. Thus, various kinds of detectors may detect the multiple activities, the combination of which is indicative of taking a diuretic in a compliant manner.

### EXAMPLE 4 ACE INHIBITORS, BETA-BLOCKERS, AND DIURETIC FOR CHF

Example 4 shows monitoring medicament compliance by detecting multiple combinations of activities associated with multiple medicaments corresponding to a medical condition. As shown in Table 4, the multiple medicaments include ACE inhibitors, Beta-blockers, and diuretic, which correspond to a medical condition of CHF. The several kinds of detectors described above in Examples 1-3 may detect the multiple combination of activities that are indicative of compliance of taking a ACE inhibitors, Beta-blockers, and diuretic. Although a single activity may not necessarily indicate compliance, the various kinds of detectors may detect multiple combinations of activities, the combination of which, are indicative of medical compliance of the multiple differing medicaments in a compliant manner.

### EXAMPLE 5 CORTICOSTEROIDS

Example 5 shows monitoring medicament compliance by detecting multiple combinations of activities associated with compliance of a Corticosteroid medicament. As shown in Table 5, several kinds of detectors may detect multiple activities that correspond to compliance of taking Corticosteroids, such as sore throat, trouble sleeping which may be detected by a motion detector, improved lung function, improved symptoms, reduced acute symptoms, reduced hospitalization need, reduced use of a rescue inhaler, and the exhaled drug or exhaled particulates which may be detected by breath detectors, either individually or in combinations of two or more. Thus, various kinds of detectors may detect the multiple activities, the combination of which, are indicative of taking a corticosteroid in a compliant manner.

### EXAMPLE 6 SHORT-ACTING BRONCHODILATORS

Example 6 shows monitoring medicament compliance by detecting multiple combinations of activities associated with compliance of a short-acting Bronchodilator medicament. As shown in Table 6, several kinds of detectors may detect multiple activities that correspond to compliance of taking short-acting Bronchodilators, such as increased heart rate which may be detected by a heart rate monitor, dry mouth which may detected by a patch, decreased appetite which may be detected by a refrigerator or stove detectors, stopping of acute asthma attack, and the exhaled drug or exhaled particulates which may be detected by breath detectors, either individually or in combinations of two or more. Thus, various kinds of detectors may detect the multiple activities, the combination of which may be indicative of taking a short-acting Bronchodilator medicament in a compliant manner.

### EXAMPLE 7 LONG-ACTING BRONCHODILATORS

Example 7 shows monitoring medicament compliance by detecting multiple combinations of activities associated with compliance of a long-acting Bronchodilator medicament. As shown in Table 7, several kinds of detectors may detect multiple activities that correspond to compliance of taking long-acting Bronchodilators, such as increased heart rate which may be detected by a heart rate monitor, dry mouth which may detected by a patch, decreased appetite which may be detected by a refrigerator or stove detectors, coughing which may be detected by a noise detector, reduced acute symptoms, reduced hospitalization need, decreased use of rescue inhaler, and the exhaled drug or exhaled particulates which may be detected by breath detectors, either individually or in combinations of two or more. Thus, various kinds of detectors may detect the multiple activities indicative of taking a long-acting Bronchodilator medicament in a compliant manner.

### EXAMPLE: 8 CORTICOSTEROIDS, SHORT-ACTING BRONCHODILATORS, AND LONG-ACTING BRONCHODILATORS FOR ASTHMA

Example 8 shows monitoring medicament compliance by detecting multiple combinations of activities associated with multiple medicaments corresponding to a medical condition. As shown in Table 8, the multiple medicaments include Corticosteroids and short and long-acting Bronchodilators, which correspond to a medical condition of asthma. The several kinds of detectors described above in examples 4-7 may detect the multiple combination of activities that are indicative of compliance of taking Corticosteroids and short and long-acting Bronchodilators. Although a single activity may not necessarily indicate compliance, the various kinds of detectors may detect multiple combinations of activities that are indicative of medical compliance of the multiple differing medicaments for asthma or another condition in a compliant manner.

### EXAMPLE 9 CHF AND ASTHMA CONDITION

Example 9 shows monitoring medicament compliance by detecting multiple combinations of activities associated with multiple medicaments corresponding to multiple differing medical conditions. As shown in Table 9, the multiple medicaments include ACE inhibitors, Beta-blockers, diuretic, corticosteroids and short and long-acting Bronchodilators, which correspond to differing medical conditions of CHF and asthma. The several kinds of detectors described above in examples 1-3 and 4-7 may detect the multiple combination of activities that are indicative of compliance of respectively taking ACE inhibitors, Beta-blockers, diuretic, Corticosteroids, short-acting Bronchodilators, and long-acting Bronchodilators. Although a single activity may not necessarily indicate compliance with multiple differing medicaments for differing medical conditions such as CHF or asthma, the various kinds of detectors may detect multiple activities, the combination of which are indicative of medical compliance of the multiple differing medicaments for differing medical conditions such as CHF and asthma.

TABLE 1

| | Detectable Side | Detectable Disease | Detectable Physiological | Physical Detection of |
|---|---|---|---|---|
| Drug Class | Effects | Abatement (or worsening) | Effects | Medicament intake |
| ACE Inhibitors | cough, swelling of | stabilization of condition; | for ingested drugs: the | Physical moving of |
| | lower legs and feet | shortness of breath while | excreted drug or drug- | medication: medicament |
| | | resting, changes in sleep or | specific metabolites | container opened, medicine |
| | | eating | excreted | bottle moved |
| | | | | Signs of drug disposal: toilet |
| | | | | flushing, garbage pail |
| | | | | movement |
| | | | | Signs of drug ingestion: water |
| | | | | running, cup movement |

TABLE 2

| | Detectable Side | Detectable Disease | Detectable Physiological | Physical Detection of |
|---|---|---|---|---|
| Drug Class | Effects | Abatement (or worsening) | Effects | Medicament intake |
| Beta-Blockers | slow pulse, tiredness, | reduced heart rate; shortness | for ingested drugs: the | Physical moving of |
| | difficulty sleeping, | of breath while resting, | excreted drug or drug- | medication: medicament |
| | sudden weight gain | changes in sleep or eating | specific metabolites | container opened, medicine |
| | | | excreted | bottle moved |
| | | | | Signs of drug disposal: toilet |
| | | | | flushing, garbage pail |
| | | | | movement |
| | | | | Signs of drug ingestion: water |
| | | | | running, cup movement |

TABLE 3

| | Detectable Side | Detectable Disease | Detectable Physiological | Physical Detection of |
|---|---|---|---|---|
| Drug Class | Effects | Abatement (or worsening) | Effects | Medicament intake |
| Diuretics | rapid weight gain, | stabilization of condition; | for ingested drugs: the | Physical moving of |
| | frequent urniation, | shortness of breath while | excreted drug or | medication: medicament |
| | extreme tiredness, | resting, changes in sleep or | drug-specific metabolites | container opened, medicine |
| | dehydration, loss of | eating | excreted | bottle moved |
| | appetite | | | Signs of drug disposal: toilet |
| | | | | flushing, garbage pail |
| | | | | movement |
| | | | | Signs of drug ingestion: water |
| | | | | running, cup movement |

TABLE 4

| | | Detectable Side | Detectable Disease | Detectable Physiological | Physical Detection of |
|---|---|---|---|---|---|
| Disease | Drug Class | Effects | Abatement (or worsening) | Effects | Medicament intake |
| CHF | ACE Inhibitors | cough, swelling of | stabilization of condition; | for ingested drugs: the | Physical moving of |
| | | lower legs and feet | shortness of breath while | excreted drug or | medication: medicament |
| | | | resting, changes in sleep or | drug-specific metabolites | container opened, medicine |
| | | | eating | excreted | bottle moved |
| | Beta-Blockers | slow pulse, tiredness, | reduced heart rate; shortness | | Signs of drug disposal: toilet |
| | | difficulty sleeping, | of breath while resting, | | flushing, garbage pail |
| | | sudden weight gain | changes in sleep or eating | | movement |
| | Diuretic | rapid weight gain, | elimination of excess fluids, | | Signs of drug ingestion: water |
| | | frequent urniation, | maintenance of constant | | running, cup movement |
| | | extreme tiredness, | weight; shortness of breath | | |
| | | dehydration, loss of | while resting, changes in | | |
| | | appetite | sleep or eating | | |

TABLE 5

| | Detectable Side | Detectable Disease | Detectable Physiological | Physical Detection of |
|---|---|---|---|---|
| Drug Class | Effects | Abatement (or worsening) | Effects | Medicament intake |
| Corticosteroids | sore throat, trouble | improved lung function, | Inhaled drugs: whole drug | Physical moving of |
| | sleeping | improved symptoms, reduced | exhaled, particulates | medication: medicament |
| | | acute symptoms, reduced | exhaled | container opened, medicine |
| | | hospitalization need; reduced | | bottle moved |
| | | use of rescue inhaler | | Signs of drug disposal: toilet |
| | | | | flushing, garbage pail |
| | | | | movement |
| | | | | Signs of drug ingestion: |
| | | | | medication movement |

TABLE 6

| | Detectable Side | Detectable Disease | Detectable Physiological | Physical Detection of |
|---|---|---|---|---|
| Drug Class | Effects | Abatement (or worsening) | Effects | Medicament intake |
| short-acting | increased heart rate, | stopping of acute asthma | for inhaled drugs: exhaled | Physical moving of |
| Bronchodilator | dry mouth, decreased | attack | drug or exhaled particulates | medication: medicament |
| | appetite | | | container opened, medicine |
| | | | | bottle moved |
| | | | | Signs of drug disposal: toilet |
| | | | | flushing, garbage pail |
| | | | | movement |
| | | | | Signs of drug ingestion: |
| | | | | medication movement |

TABLE 7

| | Detectable Side | Detectable Disease | Detectable Physiological | Physical Detection of |
|---|---|---|---|---|
| Drug Class | Effects | Abatement (or worsening) | Effects | Medicament intake |
| long-acting | increased heart rate, | reduced acute symptoms, | Inhaled drugs: whole drug | Physical moving of |
| Bronchodilators | dry throat, cough | reduced hopsitalization need; | exhaled, particulates | medication: medicament |
| | | reduced use of rescue inhaler | exhaled | container opened, medicine |
| | | | | bottle moved |
| | | | | Signs of drug disposal: toilet |
| | | | | flushing, garbage pail movement |
| | | | | Signs of drug ingestion: |
| | | | | medication movement |

TABLE 8

| | | Detectable Side | Detectable Disease | Detectable Physiological | Physical Detection of |
|---|---|---|---|---|---|
| Disease | Drug Class | Effects | Abatement (or worsening) | Effects | Medicament intake |
| Asthma | Corticosteroids | sore throat, trouble | improved lung function, | Inhaled drugs: whole drug | Physical moving of |
| | | sleeping | improved symptoms, reduced | exhaled, particulates | medication: medicament |
| | | | acute symptoms, reduced | exhaled | container opened, medicine |
| | | | hospitalization need; | | bottle moved |
| | | | decreased use of rescue | | Signs of drug disposal: toilet |
| | | | inhaler | | flushing, garbage pail |
| | short-acting | increased heart rate, | stopping of acute asthma | | movement |
| | Bronchodilators | dry mouth, decreased | attack | | Signs of drug ingestion: |
| | long-acting | appetite | | | medicament movement |
| | Bronchodilators | increased heart rate, | reduced acute symptoms, | | |
| | | dry throat, cough | reduced hopsitalization need; | | |
| | | | decreased use of rescue | | |
| | | | inhaler | | |

TABLE 9

| | | Detectable Side | Detectable Disease | Detectable Physiological | Physical Detection of |
|---|---|---|---|---|---|
| Disease | Drug Class | Effects | Abatement (or worsening) | Effects | Medicament intake |
| CHF | ACE Inhibitors | cough, swelling of | stabilization of condition; | For ingested drugs: the | Physical moving of |
| | | lower legs and feet | shortness of breath while | excreted drug or drug- | medication: medicament |
| | | | resting, changes in sleep or | specific metabolites | containeropened, medicine |
| | | | eating | Inhaled drugs: the exhaled | bottle moved |
| | Beta-Blockers | slow pulse, tiredness, | reduced heart rate; shortness | drug or exhaled particules | Signs of drugs disposal: toilet |
| | | difficulty sleeping, | of breath while resting, | | flushing, garbage pail |
| | | sudden weight gain | changes in sleep or eating | | movement |
| | Diuretics | rapid weight gain, | elimination of excess fluids, | | Signs of drug ingestion: water |
| | | frequent umiation, | maintenance of constant | | running, cup movement, |
| | | extreme tiredness, | weight; shortness of breath | | medication movement |
| | | dehydration, loss of | while resting, changes in | | |
| | | appetite | sleep or eating | | |
| Asthma | Corticosteroids | sore throat, trouble | improved lung function, | | |
| | | sleeping | improved symptoms, reduced | | |
| | | | acute symptoms, reduced | | |
| | | | hospitalization need; | | |
| | | | decreased use of rescue | | |
| | | | inhaler | | |
| | Short-acting | increased heart rate, | stopping of acute symptoms | | |
| | Bronchodilators | dry mouth, decreased | attack | edication movement | |
| | | appetite | | | |
| | Long-acting | increased heart rate, | reduced acute symptoms, | | |
| | Bronchodilators | dry throat, cough | reduced hopsitalization need; | | |
| | | | decreased use of rescue | | |
| | | | inhaler | | |

It will be apparent to those skilled in the art that various modifications and variations can be made in the method and system of the present invention without departing from the spirit or scope of the invention. Thus, it is intended that the present invention include modifications and variations that are within the scope of the appended claims and their equivalents.

While the invention has been described in detail in connection with only a limited number of aspects, it should be understood that the invention is not limited to such disclosed aspects. Rather, the invention can be modified to incorporate any number of variations, alterations, substitutions or equivalent arrangements not heretofore described, but which are commensurate with the scope of the claims. Additionally, while various embodiments of the invention have been described, it is to be understood that aspects of the invention may include only some of the described embodiments. Accordingly, the invention is not to be seen as limited by the foregoing description, but is only limited by the scope of the appended claims.

1. A medicament compliance monitoring system comprising:
a detector configured to detect an activity indicative of medical compliance of a medicament;
a transmitter in communication with the detector;
a monitoring center in communication with the transmitter;
wherein the transmitter is adapted to communicate data from the detector to the monitoring center in real-time to the occurrence of the activity indicative of compliance.

2. The system of claim 1, further comprising a communications relay panel positioned within a vicinity of the subject and in communication with the detector and the transmitter.

3. The system of claim 1, wherein the transmitter comprises at least one communication media selected from a group consisting of wired telephone, wireless telephone, two-way walkie-talkie, pager, cable, and the Internet.

4. The system of claim 1, wherein the monitoring center comprises:
a database for receiving and compiling the data on activities indicative of medical compliance.

5. The system of claim 1, wherein the monitoring center is adapted to communicate with a caregiver through at least one communication media selected from a group consisting of wired telephone, wireless telephone, pager, two way walkie-talkie, facsimile, cable, e-mail, and Internet.

6. The system of claim 1, further comprising a status report generator for generating a real-time status report upon request.

7. The system of claim 1, wherein the detector is on the subject.

8. The system of claim 1, further comprising a plurality of detectors.

9. The system of claim 8, wherein the plurality of detectors are within a vicinity of the subject.

10. The system of claim 1, wherein the detector comprises at least one detector selected from a group consisting of a motion detector, blood pressure detector, heart rate detector, medicament access detector, chemical substance detector, sleep detector, weight detector, pulse rate detector, and urinalysis detector.

11. The system of claim 1, wherein the detector is configured to detect one or more activities indicative of medical compliance selected from a group consisting of a physiological aspect, a side effect, a disease abatement, and a physical indicator.

12. The system of claim 11, wherein the physiological aspect is selected from a group consisting of an excreted form of a drug, an excreted drug-specific metabolite, an exhaled form a drug, and exhaled particulates.

13. The system of claim 11, wherein the side effect is selected from a group consisting of coughing, swelling of lower legs, swelling of feet, low pulse, tiredness, difficulty sleeping, weight gain, frequent urination, tiredness, dehydration, loss of appetite, sore throat, sleeping difficulties, increased heart rate, dry mouth, decreased appetite, dry throat, fatigue, and upset stomach.

14. The system of claim 11, wherein the disease abatement is selected from a group consisting of stabilization of a condition, shortness of breath while resting, changes in sleep, changes in eating, reduced heart rate, elimination of excess fluids, maintenance of constant weight, improved lung function, improved symptoms, reduced acute symptom, reduced hospitalization need, decreased use of rescue inhaler, and reduction of an acute attack.

15. The system of claim 1, wherein a combination of a plurality of the activities indicative of medicament compliance indicates whether the subject is compliant with a dosage intake, a dosage amount, or a dosage frequency.

16. The system of claim 1, wherein the monitoring center comprises a modeling operation configured to conclude whether the subject is compliant with the medicament based on a combination of a plurality of the activities that are indicative of medicament compliance.

17. The system of claim 16, wherein the modeling operation is configured to conclude whether the subject is compliant in a qualitative or quantitative manner.

18. The system of claim 16, wherein the modeling operation comprises at least one model selected from a group consisting of artificial intelligence, statistical modeling, and hybrid fusion techniques.

19. The system of claim 1, wherein the detector is configured to detect a plurality of activities indicative of medicament compliance.

20. The system of claim 19, wherein the plurality of activities indicative of medicament compliance correlate to a plurality of medicaments that differ from each other.

21. The system of claim 20, wherein the plurality of medicaments that differ from each other correspond to a plurality of medical conditions that differ from each other.

22. The system of claim 19, further comprising a plurality of detectors that are configured to detect the plurality of activities indicative of medicament compliance.

23. The system of claim 1, further comprising a medicament container for holding the medicament, wherein the medicament container is configured to detect removal of the medicament from the medicament container.

24. The system of claim 23, wherein the medicament container is configured to detect removal of the medicament by detecting at least one change selected from a group consisting of a change in weight, shape, size, and color.

25. The system of claim 23, wherein the medicament container is configured to detect removal of a plurality of medicaments that differ from each other.

26. The system of claim 25, wherein the medicament container is configured to detect removal of the plurality of medicaments that differ from each other by detecting a combination of changes selected from a group consisting of a change in weight, shape, size, and color.

27. The system of claim 1, wherein the monitoring center comprises a search mechanism adapted to search for patterns in the activities indicative of medicament compliance of the subject.

28. The system of claim 1, wherein a caregiver determines which activity is indicative of medicament compliance.

29. A method of monitoring medicament compliance comprising:
i) detecting an activity indicative of medical compliance of a medicament by a subject; and
ii) transmitting data of the detected activity to a monitoring center in real-time to the occurrence of the activity indicative of medicament compliance.

30. The method of claim 29, further comprising correlating the detected activity with medical compliance of the medicament.

31. The method of claim 29, further comprising correlating the detected activity with medical compliance of intaking, dosage amount, or dosage frequency of a medicament.

32. The method of claim 29, further comprising performing a modeling operation to conclude if the subject is compliant with the medicament based on a combination of a plurality of activities that are indicative of medicament compliance.

33. The method of claim 32, wherein the modeling operation comprises at least one model selected from a group consisting of artificial intelligence, statistical modeling, and hybrid fusion techniques.

34. The method of claim 29, further comprising detecting a plurality of activities indicative of medicament compliance of a medicament.

35. The method of claim 34, further comprising correlating the plurality of detected activities with medical compliance of a medicament.

36. The method of claim 35, further comprising correlating the plurality of detected activities with medical compliance of a plurality of medicaments that are different from each other.

37. The method of claim 36, further comprising correlating the plurality of detected activities with medical compliance of intaking, dosage amounts, or dosage frequencies of the plurality of medicaments.

38. The method of claim 36, further comprising performing a modeling operation to conclude if the subject is compliant with the plurality of medicaments based on a combination of a plurality of activities that are indicative of medicament compliance.

39. The method of claim 38, wherein performing the modeling operation concludes whether the subject is compliant in a qualitative or quantitative manner.

40. The method of claim 29, further comprises searching for patterns in the activities indicative of medicament compliance of the subject.

41. The method of claim 29, wherein the medicament comprises at least one medicament selected from a group consisting ACE Inhibitors, Beta-Blockers, Diuretics, Corticosteroids, short-acting Bronchodilators, long-acting Bronchodilators, and Statins.

42. The method of claim 29, wherein the medicament comprises at least one medicament capable of treating a condition selected from a group consisting asthma, cholesterol, and CHF.

43. The method of claim 29, further comprising providing the medicament in a medicament container, wherein the medicament container is operable to detect removal of the medicament.

44. The method of claim 43, wherein the medicament container is operable to detect removal of a medicament by detecting at least one change selected from a group consisting of a change in a weight, shape, size, and color.

45. The method of claim 43, wherein the medicament container is operable to detect removal of a plurality of medicaments that differ from each other.

46. The method of claim 45, wherein the medicament container is operable to detect removal of the plurality of medicaments that differ from each other by detecting a combination of changes selected from a group consisting of weight, shape, size, and color.

47. The method of claim 29, further comprising detecting a base-line pre-medicament detection, from the subject, of the activity indicative of medicament compliance.

48. The method of claim 46, further comprising comparing the base-line pre-medicament detection with the detection concurrent with a medicament.

49. The method of claim 29, further comprising detecting a post-medicament detection.

50. A method of monitoring medicament compliance of a subject comprising:
i) providing an activity detector, wherein the activity is indicative of medical compliance of a medicament;
ii) collecting data from the activity detector;
iii) providing a transmitter in communication with the detector;
wherein the transmitter communicates the data from the activity detector to a monitoring center in real-time to the occurrence of the activity indicative of medicament compliance.

51. A medicament container comprising:
a compartment configured to hold a medicament; and
wherein the compartment comprises a sensor configured to detect removal of the medicament.

52. The medicament container of claim 51, wherein the sensor is in communication with a transmitter adapted to communicate data from the detector to the monitoring center in real-time to the occurrence of the activity indicative of compliance.

53. The medicament container of claim 51, further comprising a plurality of compartments.

54. The medicament container of claim 51, wherein the medicament container comprises a circuit.

55. The medicament container of claim 51, wherein the compartment comprises a cover and the sensor is configured to detect removal of the medicament in correlation with an opening of the cover.

56. A medicament compliance monitoring system comprising:
a plurality of detectors configured to detect a plurality of activities indicative of medical compliance of a medicament; and
a monitoring center for receiving data from the plurality of detectors in real-time to the occurrence of the plurality of activities indicative of compliance.
